(19) United States
(12) Patent Application Publication
(10) Pub. No.: US 2025/0107688 A1
(43) Pub. Date: Apr. 3, 2025

(54) INFORMATION PROCESSING METHOD, INFORMATION PROCESSING SYSTEM, AND PROGRAM

(71) Applicant: Sony Group Corporation, Tokyo (JP)

(72) Inventor: Masaya TAKEMOTO, Kanagawa (JP)

(73) Assignee: Sony Group Corporation, Tokyo (JP)

(21) Appl. No.: 18/728,898

(22) PCT Filed: Jan. 12, 2023
(86) PCT No.: PCT/JP2023/000541

§ 371 Date: Jul. 15, 2024

(30) Foreign Application Priority Data
Jan. 27, 2022 (JP) 2022-010940

(51) Int. Cl. A61B 1/00

(52) U.S. Cl. CPC A61B 1/00009; A61B 1/00018

(57) ABSTRACT

The present technology relates to an information processing method, an information processing system, and a program that enable appropriate quality control of surgical videos according to a situation.
An information processing method according to one aspect of the present technology includes: controlling, on the basis of a selection status of a video by a user viewing a plurality of surgical videos using a reception device, the plurality of surgical videos including a video captured in a surgical operation room, and a status of a communication line, quality of each of a plurality of surgical videos; and transmitting the surgical videos the quality of which has been controlled to the reception device via an information processing server on a network. The present technology can be applied to a case where a surgeon in a remote place instructs, as a medical adviser, a surgeon in a surgical operation room how to perform the surgery.

### TECHNICAL FIELD

The present technology relates to an information processing method, an information processing system, and a program, and particularly relates to an information processing method, an information processing system, and a program that enable appropriate quality control of surgical videos according to a situation.

### BACKGROUND ART

Various services including medical services are more and more cloud-based. Against the background of the recent labor shortage, even in a surgery, a method has been developed in which a skilled surgeon provides instructions from outside the surgical operation room by viewing a surgical video distributed via a cloud.

Patent Document 1 discloses a technique in which, in communication between an emergency scene and an emergency center, when a communication bandwidth is not sufficient to transmit both video and voice from the emergency scene, transmission of the video is temporarily stopped and transmission of patient data is prioritized.

### CITATION LIST

### Patent Document

Patent Document 1: Japanese Patent Application Laid-Open No. 2018-149378

### SUMMARY OF THE INVENTION

### Problems to be Solved by the Invention

In a case where a video is distributed via a cloud, it is assumed that a delay occurs. In addition, in video distribution, a trade-off usually exists between video quality and real-time property. For example, in order to maintain the real-time property, the video quality needs to be reduced.

However, as described above, in a case where a medical advisor instructs how to perform the surgery from a remote place, it is important to distribute a high-quality medical video to the medical advisor in real time.

The present technology has been made in view of such a situation, and an object thereof is to enable appropriate quality control of a surgical video according to a situation.

### Solutions to Problems

An information processing method according to one aspect of the present technology includes: controlling, on the basis of a selection status of a video by a user viewing a plurality of surgical videos using a reception device, the plurality of surgical videos including a video captured in a surgical operation room, and a status of a communication line, quality of each of a plurality of the surgical videos, and transmitting the surgical videos the quality of which has been controlled to the reception device via an information processing server on a network.

In the present technology, on the basis of a selection status of a video by a user viewing a plurality of surgical videos using a reception device, the plurality of surgical videos including a video captured in a surgical operation room, and a status of a communication line, quality of each of a plurality of the surgical videos is controlled, and the surgical videos the quality of which is controlled are transmitted to the reception device via an information processing server on a network.

### BRIEF DESCRIPTION OF DRAWINGS

FIG. 1 is a diagram illustrating an example of an information processing system according to an embodiment of the present technology.

FIG. 2 is a diagram illustrating a configuration example of a surgical operation room system.

FIG. 3 is a block diagram illustrating a functional configuration example of each device included in the information processing system.

FIG. 4 is a diagram illustrating a display example of a surgical video in a reception device.

FIG. 5 is a sequence diagram explaining the procedure of each device included in the information processing system.

FIG. 6 is a diagram illustrating a specific example of a status of a communication line.

FIG. 7 is a diagram illustrating a first specific example of quality control of a surgical video.

FIG. 8 is a diagram illustrating a second specific example of quality control of a surgical video.

FIG. 9 is a diagram illustrating a third specific example of quality control of a surgical video.

FIG. 10 is a block diagram illustrating a configuration example of hardware of a computer.

### MODE FOR CARRYING OUT THE INVENTION

Hereinafter, modes for carrying out the present technology will be described. The description will be given in the following order.
1. Overview of present technology
2. Functional configuration of information processing system
3. Operation of information processing system
4. Example of quality control
5. Specific examples of quality control
6. Modifications

### Overview of Present Technology

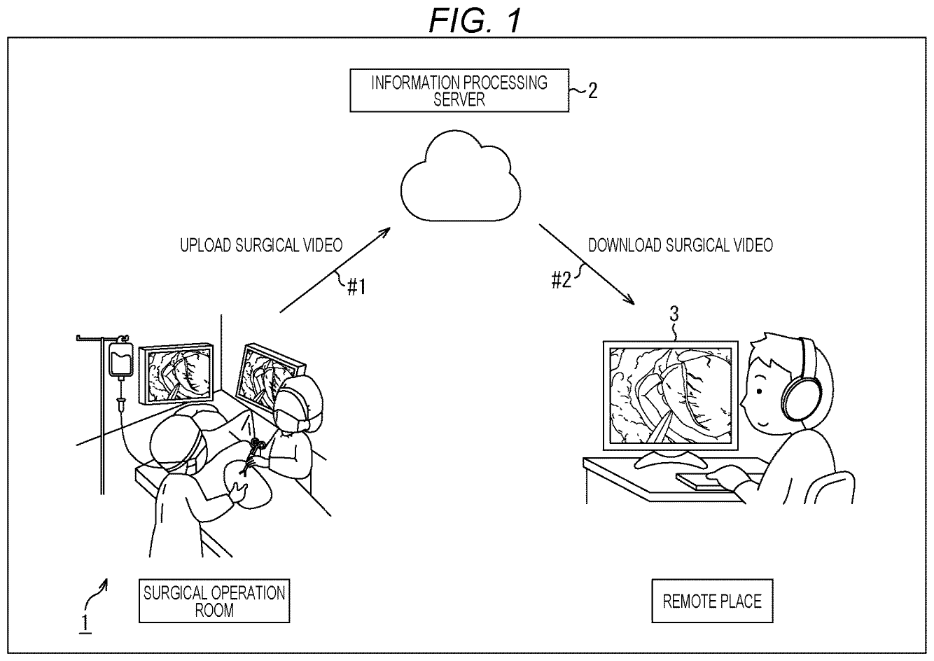

FIG. 1 is a diagram illustrating an example of an information processing system according to an embodiment of the present technology.

An information processing system according to an embodiment of the present technology includes a surgical operation room system 1, an information processing server 2, and a reception device 3. Each configuration is connected via a network such as the Internet.

The surgical operation room system 1 is provided in a medical facility having a surgical operation room, such as a hospital. As will be described later, the surgical operation room system 1 includes a plurality of cameras such as a camera that images a surgical site and a camera that images a state in the surgical operation room.

The information processing server 2 is a server (cloud server) on a network. Communication is performed via the Internet both between the surgical operation room system 1 and the information processing server 2 and between the information processing server 2 and the reception device 3.

The reception device 3 is a device located in a place distant from the surgical operation room, such as the user's home. The reception device 3 includes a PC, a tablet terminal, and the like. For example, the reception device 3 is operated by a surgeon who is in a remote place as a user. The reception device 3 may be used at various places other than the user's home as long as the reception device 3 is located distant from the surgical operation room.

As indicated by an arrow #1 in FIG. 1, surgical videos such as a video of a surgery performed in the surgical operation room and a video of a screen showing patient's vital signs are uploaded to the information processing server 2 in real time. As indicated by an arrow #2, the uploaded surgical videos are transmitted (downloaded) to the reception device 3 via the information processing server 2 in real time.

The surgical video transmitted to the reception device 3 is displayed on a display of the reception device 3. The user of the reception device 3 can check the content of the surgery performed in the surgical operation room by viewing the surgical video displayed on the display, and give instructions to the surgeon or the like in the surgical operation room in real time. Video or audio data indicating the content of the instruction is transmitted from the reception device 3 to the surgical operation room system 1 via the information processing server 2, and is output in the surgical operation room.

As described above, the information processing system in FIG. 1 is used, for example, in a case where a surgeon in a remote place (the user of the reception device 3) instructs, as a medical advisor, a surgeon in a surgical operation room how to perform the surgery.

As will be described in detail later, the user of the reception device 3 can select a video to be viewed in high quality from among a plurality of surgical videos uploaded from the surgical operation room system 1. The information indicating the selection status of the video by the user is transmitted to the surgical operation room system 1 and used for controlling the quality of the surgical videos to be uploaded.

In the surgical operation room system 1, the quality of the surgical video is controlled on the basis of the selection status of a video by the user and the status of the communication line. The quality is controlled by adjusting the resolution or the frame rate of each surgical video.

For example, the quality of the surgical videos to be uploaded is controlled such that the video selected by the user is transmitted in high quality and the video not selected by the user is transmitted in low quality. As a result, even in a case where the communication line is in a status in which not all the surgical videos can be transmitted in high quality, both the real-time property and the quality of the surgical video can be maintained.
Configuration of surgical operation room system 1

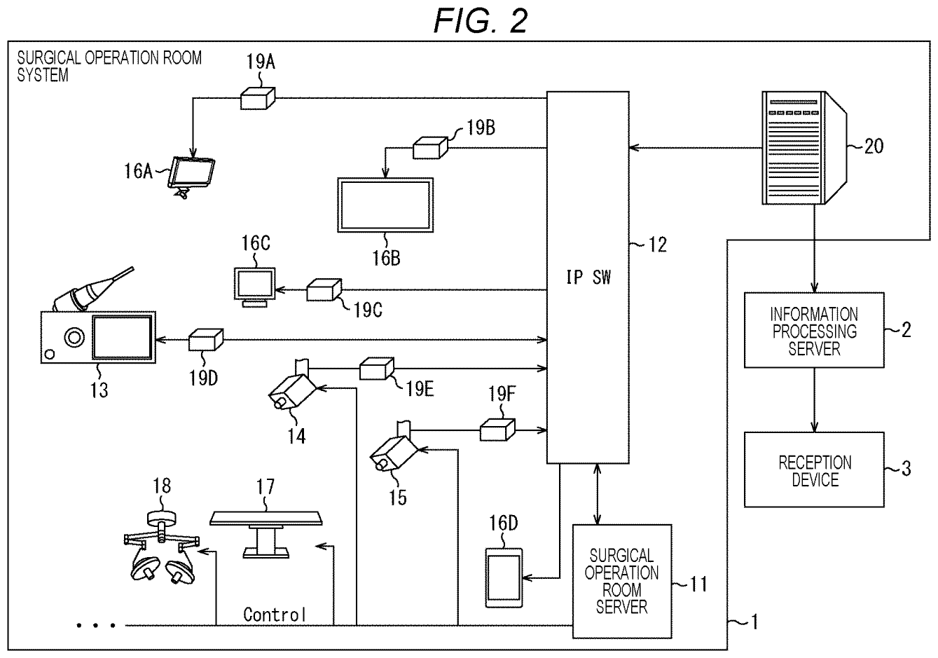

FIG. 2 is a diagram illustrating a configuration example of the surgical operation room system 1 in FIG. 1.

In the surgical operation room system 1, device groups installed in a surgical operation room are connected via a surgical operation room server 11 and an IP switch (SW) 12 so as to be able to cooperate with each other. The surgical operation room system 1 includes an Internet protocol (IP) network capable of transmitting and receiving 4K/8K images, and input and output images and control information directed to each device are transmitted and received via the IP network.

Various devices are installed in the surgical operation room. In an example in FIG. 2, a device group 13 including various devices used for endoscopic surgery, a ceiling camera 14 that images hands of a surgeon, a surgical field camera 15 that images a state of the entire surgical operation room, display devices 16A to 16D, a patient bed 17, and a light 18 are illustrated. Both the ceiling camera 14 and the surgical field camera 15 are installed on the ceiling of the surgical operation room. In addition to the illustrated endoscope, various medical devices that acquire images, such as a master-slave endoscopic surgical robot and an X-ray imaging device, may be included in the device group 13.

The display devices 16A to 16C, the device group 13, the ceiling camera 14, and the surgical field camera 15 are connected to the IP SW 12 via IP converters 19A to 19F, respectively. Hereinafter, in a case where the IP converters 19A to 19F and the like are not especially distinguished from one another, they are simply referred to as IP converters 19.

The IP converters 19D, 19E, and 19F are IP converters 19 on an input source side (camera side) that input images and they perform IP conversion on an image from the respective medical imaging devices (endoscope, surgical microscope, X-ray imaging device, surgical field camera, pathological imaging device and the like) and transmit the converted image over a network. The IP converters 19A to 19C are IP converters 19 on an image output side (monitor side) that output images, and they convert the image transmitted via the network into a format specific to a monitor and output the converted image. The IP converters 19 on the input source side function as encoders, and the IP converters 19 on the image output side function as decoders. The input source includes, for example, a video source.

The IP converters 19 can have various image processing functions. For example, the IP converters 19 can have a function of performing resolution conversion processing according to an output destination, a function of performing rotation correction and camera shake correction on an endoscopic image, a function of performing object recognition processing, and the like.

These image processing functions may be specific to the connected medical imaging devices, or may be externally upgradable. The IP converters 19 on the image output side (monitor side) can also perform processing such as composition of a plurality of images (PinP processing and the like) and superimposition of annotation information.

A protocol conversion function of the IP converters 19 is a function of converting a received signal into a converted signal that conforms to a communication protocol communicable on a network such as the Internet, for example. Any communication protocol may be set as the communication protocol. Furthermore, a protocol-convertible signal received by the IP converters 19 is a digital signal such as an image signal or a pixel signal, for example. The IP converters 19 may be incorporated in the devices on the input source side or devices on the image output side.

The device group 13 belongs to, for example, an endoscopic surgery system, and includes an endoscope, a display device that displays an image captured by the endoscope and the like. In contrast, the display devices 16A to 16D, the patient bed 17, and the light 18 are devices provided in the surgical operation room separately from the endoscopic surgery system. Each of the devices used for surgery or diagnosis are also referred to as medical devices. The surgical operation room server 11 and/or the IP SW 12 cooperatively control the operations of these medical devices. Furthermore, in a case where a medical image acquisition device such as a surgical robot (surgical master-slave) system and an X-ray imaging device is included in the surgical operation room, such devices are also connected as the device group 13.

Here, out of the devices included in the surgical operation room system 1, the device group 13, the ceiling camera 14, and the surgical field camera 15 are devices (hereinafter, also referred to as source devices) that have a function of transmitting information to be displayed during surgery (hereinafter, also referred to as display information). Furthermore, the display devices 16A to 16D are devices that output the display information (hereinafter, also referred to as destination devices).

The surgical operation room server 11 centrally controls processing performed in the surgical operation room system 1.

The surgical operation room server 11 has a function of controlling the operations of the source devices and the destination devices, obtaining the display information from the source devices, transmitting the display information to the destination devices, and allowing the destination devices to display or record the display information. The display information includes various images captured during surgery, various pieces of information regarding the surgery (for example, physical information of a patient, and information regarding a past examination result and a surgical method) and the like.

Specifically, information regarding an image of a surgical site in the body cavity of the patient captured by the endoscope is transmitted to the surgical operation room server 11 as the display information from the device group 13. In addition, information regarding an image of the hands of the surgeon captured by the ceiling camera 14 is transmitted to the surgical operation room server 11 as the display information from the ceiling camera 14. Furthermore, information regarding an image showing the state of the entire surgical operation room captured by the surgical field camera 15 is transmitted to the surgical operation room server 11 as the display information from the surgical field camera 15. In a case where the surgical operation room system 1 includes other devices having an imaging function, the surgical operation room server 11 may acquire information regarding images captured by the other devices as the display information from the other devices.

The surgical operation room server 11 causes at least any one of the display devices 16A to 16D, which are destination devices, to display the acquired display information (in other words, an image captured during surgery and various types of information regarding the surgery). In the example in FIG. 2, the display device 16A is a display device installed on the ceiling of the surgical operation room in a suspended manner, and the display device 16B is a display device installed on a wall surface of the surgical operation room. The display device 16C is a display device installed on a desk in the surgical operation room, and the display device 16D is a mobile device (for example, a tablet personal computer (PC) or a smartphone) having a display function.

As will be described in detail later, the surgical operation room server 11 also controls the quality of the surgical video to be transmitted to the reception device 3 via the information processing server 2.

The IP SW 12 is configured as one of input/output controllers that control input and output of the image signal to and from connected devices. For example, the IP SW 12 controls input and output of the image signal under the control of the surgical operation room server 11. The IP SW 12 controls high-speed transfer of the image signal between devices located on the IP network.

The surgical operation room system 1 may include a device located outside the surgical operation room. The device located outside the surgical operation room is, for example, a server connected to a network constructed inside or outside the hospital, a PC used by a medical staff, a projector installed in a conference room of the hospital and the like. In a case where such an external device is present outside the hospital, the surgical operation room server 11 can also allow a display device of another hospital to display the display information, via a video conference system or the like, for telemedicine.

An external server 20 communicates with, for example, an in-hospital server outside the surgical operation room and the information processing server 2 that is a cloud server. The image information in the surgical operation room is transmitted to the information processing server 2 via the external server 20. The data to be transmitted may be a surgical video itself captured by the endoscope and the like, metadata extracted from images, data indicating an operation status of a connected device and the like.

In the surgical operation room system 1, the IP network may be configured as a wired network, or a part or all of the IP network may be configured as a wireless network. For example, the IP converters 19 on the input source side having a wireless communication function may transmit a received image to the IP converters 19 on the image output side via a wireless communication network such as a fifth-generation mobile communication system (5G) or a sixth-generation mobile communication system (6G).

### Functional Configuration of Information Processing System

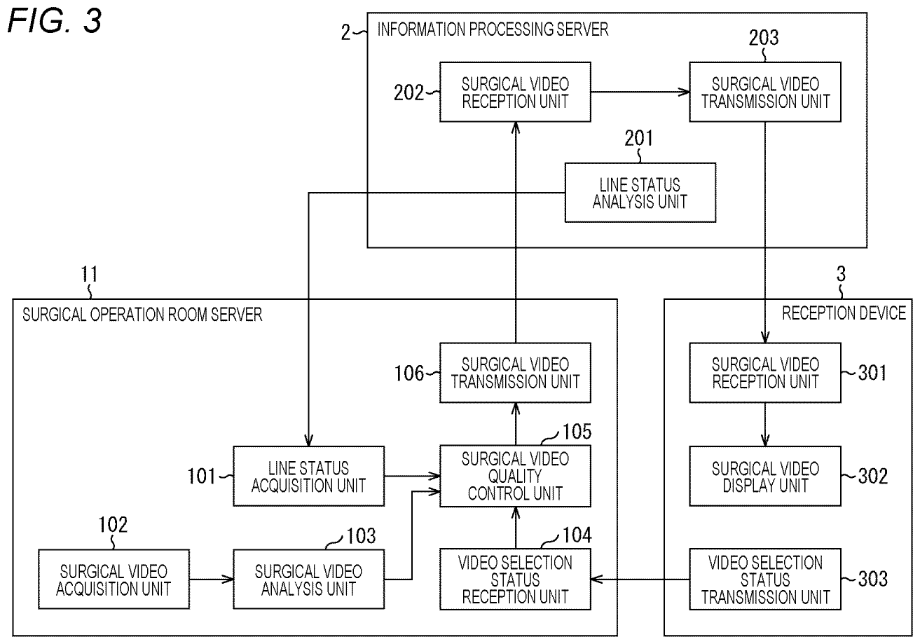

FIG. 3 is a block diagram illustrating a functional configuration example of each device included in the information processing system in FIG. 1. Each function illustrated in FIG. 3 is implemented by the CPU of a computer included in each device by executing a predetermined program.
Functional configuration of surgical operation room server 11

The surgical operation room server 11 includes a line status acquisition unit 101, a surgical video acquisition unit 102, a surgical video analysis unit 103, a video selection status reception unit 104, a surgical video quality control unit 105, and a surgical video transmission unit 106.

The line status acquisition unit 101 acquires information indicating the status of the communication line transmitted from the information processing server 2. The information indicating the status of the communication line is output to the surgical video quality control unit 105.

The surgical video acquisition unit 102 acquires surgical videos transmitted from a plurality of cameras installed in the surgical operation room. The surgical videos acquired by the surgical video acquisition unit 102 include, for example, a video captured by the endoscope of the device group 13 in FIG. 2, a video of the hands of the surgeon captured by the ceiling camera 14, a video of the entire surgical operation room captured by the surgical field camera 15, and a video of a screen indicating the vital signs of the patient. The surgical video acquired by the surgical video acquisition unit 102 is output to the surgical video analysis unit 103.

The surgical video analysis unit 103 analyzes the surgical video supplied from the surgical video acquisition unit 102. The surgical video is analyzed by using, for example, an inference model generated in advance by machine learning using a plurality of videos showing various surgical acts as learning data. By inputting the surgical video to the inference model, information representing the content of the surgery and the importance of the content of the surgery is output. The information of the analysis result by the surgical video analysis unit 103 is output to the surgical video quality control unit 105 together with the surgical video.

The video selection status reception unit 104 receives video selection information that is information indicating which surgical video is selected in the reception device 3. In the reception device 3, for example, selection of a surgical video to be viewed in high quality, selection of which surgical video is to be set as a primary video or a secondary video, and the like are performed. In accordance with the selection of the surgical video, the video selection information is transmitted from the reception device 3 to the surgical operation room server 11. The video selection information received by the video selection status reception unit 104 is output to the surgical video quality control unit 105.

The surgical video quality control unit 105 controls the quality of the surgical video to be transmitted to the reception device 3 on the basis of the information supplied from each of the line status acquisition unit 101, the surgical video analysis unit 103, and the video selection status reception unit 104. A specific example of how to control the quality will be described later. The surgical video the quality of which is controlled by the surgical video quality control unit 105 is output to the surgical video transmission unit 106.

The surgical video transmission unit 106 transmits (uploads) the quality-controlled surgical video supplied from the surgical video quality control unit 105 to the information processing server 2 via, for example, the external server 20 (FIG. 2).

In this example, all the functional units of the line status acquisition unit 101, the surgical video acquisition unit 102, the surgical video analysis unit 103, the video selection status reception unit 104, the surgical video quality control unit 105, and the surgical video transmission unit 106 are implemented by the surgical operation room server 11, but at least some of the functional units may be implemented in another device in the surgical operation room system 1. For example, the function of the surgical video quality control unit 105 that performs image processing on the surgical video can be implemented by the IP converters 19.
Functional configuration of information processing server 2

The information processing server 2 includes a line status analysis unit 201, a surgical video reception unit 202, and a surgical video transmission unit 203. The functional units of the information processing server 2 may be implemented by a plurality of computers.

The line status analysis unit 201 communicates with, for example, the external server 20 of the surgical operation room system 1, and analyzes the status of the communication line between the information processing server 2 and the surgical operation room system 1. Furthermore, the line status analysis unit 201 communicates with the reception device 3 and analyzes the status of the communication line between the information processing server 2 and the reception device 3. The line information that is information indicating the analysis result by the line status analysis unit 201 is transmitted to the surgical operation room server 11.

The surgical video reception unit 202 receives the surgical video transmitted from the surgical operation room server 11. The surgical video received by the surgical video reception unit 202 is output to the surgical video transmission unit 203.

The surgical video transmission unit 203 transmits the surgical video supplied from the surgical video reception unit 202 to the reception device 3.
Functional configuration of reception device 3 The reception device 3 includes a surgical video reception unit 301, a surgical video display unit 302, and a video selection status transmission unit 303. The surgical video reception unit 301 receives the surgical video transmitted from the information processing server 2.

The surgical video received by the surgical video reception unit 301 is output to the surgical video display unit 302.

The surgical video display unit 302 displays the surgical video supplied from the surgical video reception unit 301 on the display of the reception device 3.

The video selection status transmission unit 303 receives the selection of the surgical video by the user, and transmits video selection information corresponding to the selected content to the surgical operation room server 11. The video selection information may be transmitted via the information processing server 2, or may be directly transmitted from the reception device 3 to the surgical operation room server 11.

Selection of a partial region of the surgical video may be performed by the user. In this case, information indicating the region selected by the user is included in the video selection information and transmitted to the surgical operation room server 11. In this manner, various types of information regarding the selection of the surgical video are included in the video selection information.

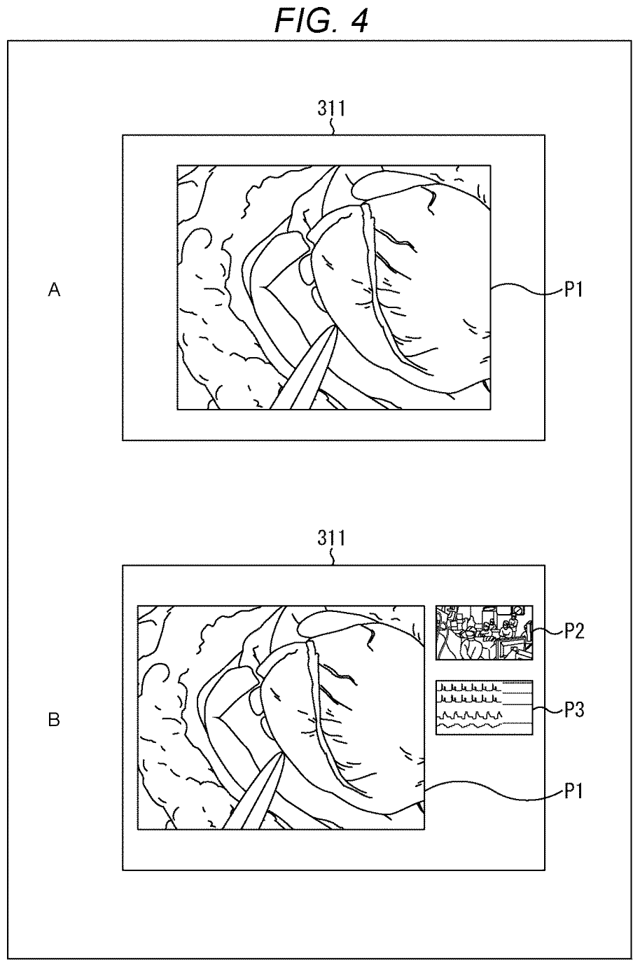

FIG. 4 is a diagram illustrating a display example of a surgical video in the reception device 3.

As illustrated in FIG. 4, the surgical video transmitted from the surgical operation room server 11 is displayed in various formats on a display 311 of the reception device 3. The screen illustrated in A of FIG. 4 is a screen displaying a surgical video P1 that is a video of the surgical site in an enlarged manner. The screen illustrated in A of FIG. 4 is displayed when, for example, the user selects viewing of only the video of the surgical site in high quality in a larger size.

By displaying the surgical video P1 as illustrated in A of FIG. 4, the user can intensively view only the video of the surgical site.

The screen illustrated in B of FIG. 4 is a screen displaying the surgical video P1 on the left side in an enlarged manner and a surgical video P2 and a surgical video P3 on the right side in a smaller size. The surgical video P2 is a video showing a state of the entire surgical operation room. The surgical video P3 is a video of a screen showing vital signs of the patient. The screen illustrated in B of FIG. 4 is displayed when, for example, the surgical video P1 is set as the primary video and the surgical videos P2 and P3 are set as the secondary videos. The primary video is the surgical video with the highest priority, and the secondary video is the surgical video with a lower priority than the primary video. The user can also set the priority for each surgical video.

By displaying each surgical video as illustrated in B of FIG. 4, the user can view the video of the surgical site while checking the state of the entire surgical operation room and the vital signs of the patient.

In this manner, the user can cause the surgical videos to be displayed in various formats such as causing a video that the user wants to view attentively to be displayed in an enlarged manner.

### Operation of Information Processing System

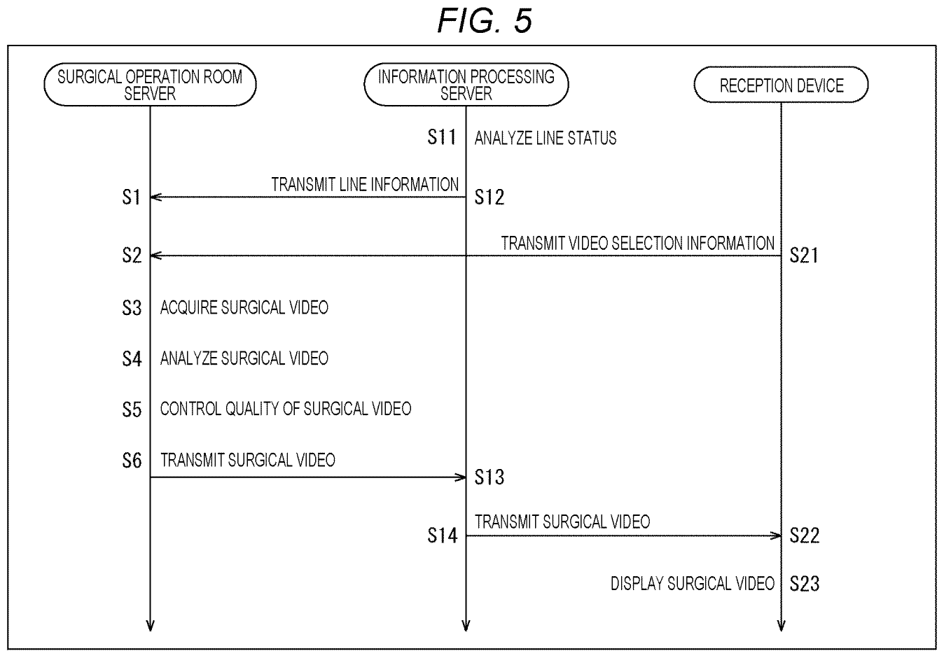

Here, the procedure of each of the surgical operation room server 11, the information processing server 2, and the reception device 3 having the above-described configuration will be described with reference to the sequence diagram in FIG. 5.

In step S11, the line status analysis unit 201 of the information processing server 2 analyzes the status of the communication line, and in step S12, transmits the line information to the surgical operation room server 11.

In step S1, the line status acquisition unit 101 of the surgical operation room server 11 receives and acquires the line information transmitted from the information processing server 2.

In step S21, the video selection status transmission unit 303 of the reception device 3 accepts the selection by the user, and transmits the video selection information to the surgical operation room server 11. The surgical video may be selected while the user is viewing the video transmitted from the surgical operation room server 11.

In step S2, the video selection status reception unit 104 of the surgical operation room server 11 receives the video selection information transmitted from the reception device 3.

In step S3, the surgical video acquisition unit 102 receives and acquires surgical videos transmitted from a plurality of cameras installed in the surgical operation room.

In step S4, the surgical video analysis unit 103 analyzes the surgical videos.

In step S5, the surgical video quality control unit 105 controls the quality of the surgical videos on the basis of the line information and the video selection information.

In step S6, the surgical video transmission unit 106 transmits the quality-controlled surgical videos to the information processing server 2.

In step S13, the surgical video reception unit 202 of the information processing server 2 receives the surgical videos transmitted from the surgical operation room server 11.

In step S14, the surgical video transmission unit 203 transmits the surgical videos to the reception device 3.

In step S22, the surgical video reception unit 301 of the reception device 3 receives the surgical videos transmitted from the information processing server 2.

In step S23, the surgical video display unit 302 displays the surgical videos on the display.

According to the above processing, even in a case where the communication line is in a status in which not all the surgical videos can be transmitted in high quality, the surgical video especially selected by the user can be transmitted with both the real-time property and the quality being maintained.

### Example of Quality Control

An example of quality control by the surgical video quality control unit 105 will be described. The quality may be controlled by combining two or more of the following processes.
First example (example of transmitting only surgical video selected by user in high quality)

The quality of the surgical video is controlled by reducing the quality of the surgical videos other than the surgical video selected by the user.

In this case, the surgical video selected by the user is transmitted in a high-quality state, and the other surgical videos are transmitted in a lower-quality state. For example, only the surgical video selected as the primary video by the user is transmitted in a high-quality state. The quality of the surgical video is reduced by, for example, reducing at least one of the resolution and the frame rate of the surgical video.
Second example (example of transmitting only the region selected by the user in high quality)

In a case where the user selects a partial region of a certain surgical video, the quality of the surgical video is controlled by reducing the quality of the region other than the region selected by the user.

In this case, the region selected by the user in a certain surgical video is transmitted in a high-quality state, and the other region is transmitted in a lower-quality state. For example, only a region showing an organ that is the target of the surgery is transmitted in a high-quality state. Reducing the quality of a partial region is achieved by, for example, reducing the resolution of the region other than the region selected by the user.

The region selected by the user may be cut out by cropping each frame constituting the surgical video, and the video of the cut-out region may be transmitted as the quality-controlled surgical video. The video obtained by cutting out a partial region is a video having a lower resolution than that of the original video, and thus is a video having a reduced quality.
Third example (example of controlling quality on the basis of priority)

In a case where the user selects two surgical videos as the primary video and the secondary video, the quality of the surgical video is controlled on the basis of the priority set according to the selection by the user.

In this case, the primary video, which is the surgical video with high priority, is transmitted in a high-quality state, and the secondary video, which is the surgical video with lower priority than the primary video, is transmitted in a lower-quality state.
Fourth example (example of selecting quality reduction method)

The user may be allowed to select which one of the resolution and the frame rate is to be reduced as to how to reduce the quality of the secondary video. The user may be allowed to select to reduce both the resolution and the frame rate.

In this case, information indicating the content of the user's selection regarding how to reduce the quality is included in, for example, the video selection information and transmitted from the reception device 3 to the surgical operation room server 11. Instead of the user, another person such as a surgeon in the surgical operation room may be allowed to select the way of reducing.
Fifth example (example of not transmitting surgical videos other than surgical video having high priority)

The quality of the surgical video is controlled by not transmitting surgical videos other than a certain number of surgical videos having high priority set according to the selection by the user.

For example, in a case where there are three or more types of surgical videos and one primary video and one secondary video are selected, only the primary video and the secondary video that are high-priority surgical videos are transmitted in high quality. Surgical videos other than the primary video and the secondary video are excluded from the transmission target as surgical videos having lower priority. The user may select the number of surgical videos to be transmitted in high quality. As the number of videos to be transmitted is reduced, the quality of the entire surgical video is reduced.
Sixth example (example of combining and transmitting a plurality of surgical videos)

The quality of the surgical video is controlled by combining a certain number of surgical videos having high priority set according to the selection by the user into a single video.

For example, videos are combined by side-by-side processing in which each frame of the primary video and each frame of the secondary video are arranged side by side, or by PinP processing in which each frame of the secondary video in a reduced size is superimposed on the corresponding frame of the primary video. The combined surgical video becomes a video having a lower resolution than that of the original video, and thus has a reduced quality. The user may be allowed to select the number of surgical videos to be combined.

The surgical videos may be combined in the information processing server 2 instead of the surgical operation room server 11. In this case, a processing unit having a function similar to that of the surgical video quality control unit 105 is implemented by the information processing server 2.
Seventh example (example of controlling quality on the basis of attribute information of surgical video) The priority of the surgical video is set on the basis of medical device information included in the attribute information of the surgical video, and the quality of the surgical video is controlled on the basis of the set priority. For example, attribute information such as digital imaging and communications in medicine (DICOM) is added to the surgical video output from the device group 13 used for endoscopic surgery. DICOM includes medical device information indicating the type of medical device that is the output source of the video, and the like.

In this case, the priority of the surgical video is automatically set by the surgical operation room server 11. For example, higher priority is set to a surgical video captured by an endoscope.
Eighth example (example of controlling quality on the basis of surgical status)

The content of the surgical video is analyzed by the surgical video analysis unit 103, and the quality of the surgical video is controlled on the basis of the status of the surgery. The content of the surgical video is analyzed using, for example, an inference model in which each frame of the surgical video is input and information indicating the status of the surgery is output. On the basis of the output of the inference model, the importance of the content of the surgery is specified. For example, a surgical video showing a state of incision of an important organ is specified as an image with higher importance.

In this case, the surgical video specified as the video with higher importance is transmitted in a high-quality state as it is. As for the surgical video specified as a video with lower importance, the resolution is reduced and the surgical video is transmitted in a lower-quality state.
Ninth example (example of controlling quality on the basis of communication line status)

The quality of the surgical video is controlled on the basis of the status of the communication line.

For example, in a case where the communication line is in a status in which not all the surgical videos can be transmitted in high quality, the surgical video selected by the user is transmitted in high quality. As for the surgical videos not selected by the user, the resolution is reduced and the surgical videos are transmitted in a lower quality.
Tenth example (example of controlling quality on the basis of both surgical status and communication line status)

The quality of the surgical video is controlled on the basis of both the status of the surgery and the status of the communication line.

For example, in a case where the communication line is in a status in which not all the surgical videos can be transmitted in high quality, the surgical video specified as a video with higher importance is transmitted in a high-quality state as it is. As for the surgical video specified as a video with lower importance, the resolution is reduced and the surgical video is transmitted in a lower-quality state.

### Specific Examples of Quality Control

Here, specific examples of quality control by the surgical video quality control unit 105 will be described.
First specific example (specific example of ninth quality control described above)

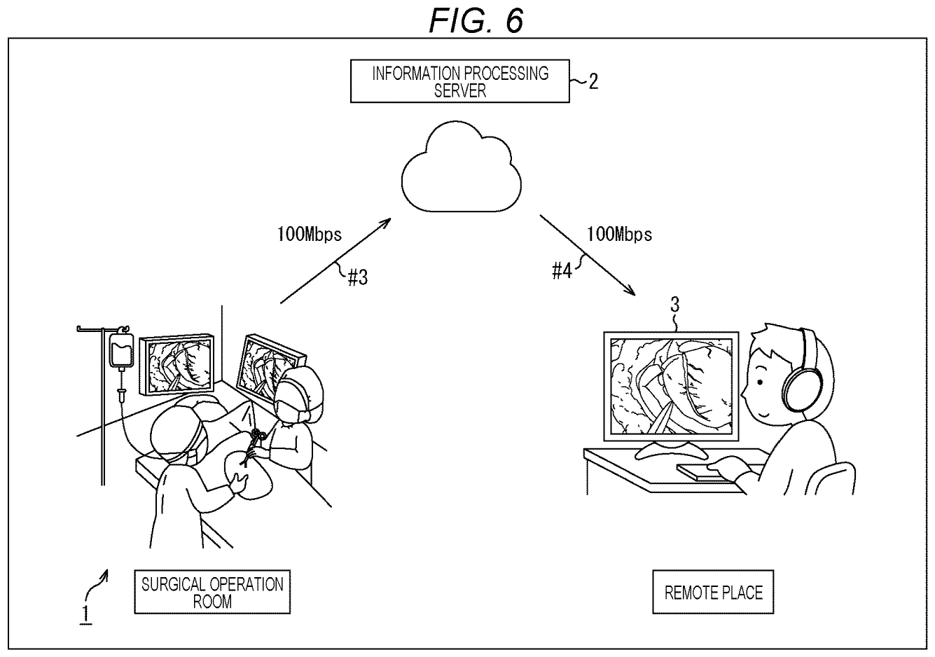

FIG. 6 is a diagram illustrating a specific example of the status of the communication line.

A case where the status (transmission rate) of the communication line between the information processing server 2 and the surgical operation room system 1 has been analyzed to be 100 Mbps as indicated by an arrow #3 in FIG. 6 and the status of the communication line between the information processing server 2 and the reception device 3 has been analyzed to be 100 Mbps as indicated by an arrow #4 will be described. The status of the communication line is analyzed by the line status analysis unit 201. The line information is transmitted from the information processing server 2 to the surgical operation room system 1, and is acquired by the line status acquisition unit 101 of the surgical operation room server 11.

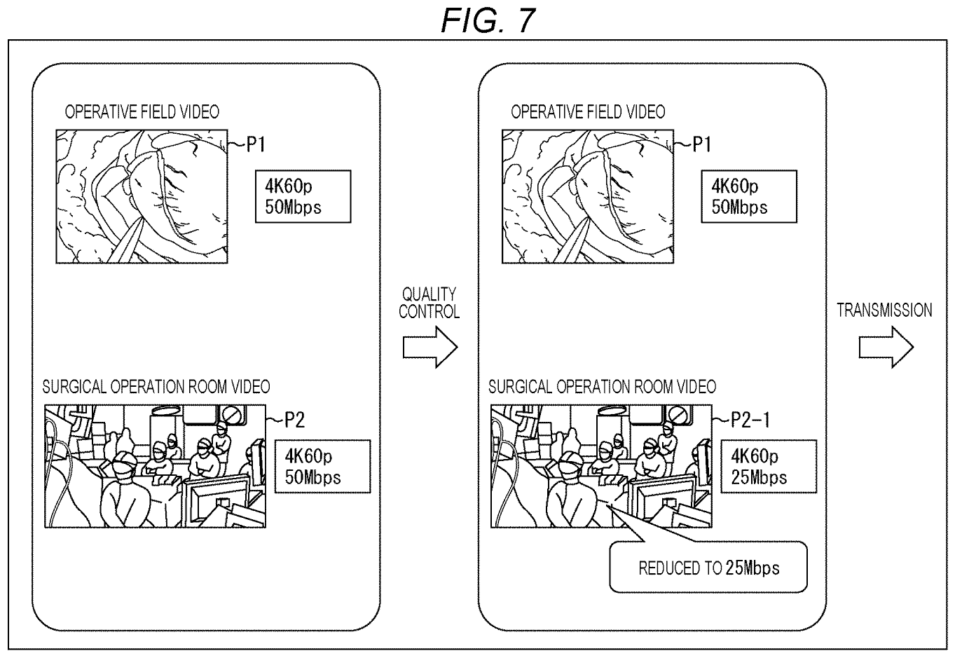

Furthermore, as illustrated on the left side of FIG. 7, it is assumed that a surgical video P1 that is an operative field video and a surgical video P2 that is a surgical operation room video have been acquired by the surgical video acquisition unit 102. The surgical video P1 and the surgical video P2 each have the resolution of 4K, the frame rate of 60 p, and the bit rate of 50 Mbps. For example, the surgical video P1 is the primary video, and the surgical video P2 is the secondary video.

Since the sum of the bit rate of the surgical video P1 and the bit rate of the surgical video P2 is equal to the transmission rate between the three places, it is difficult, in this situation, to transmit the surgical video P1 and the surgical video P2 while keeping the original quality.

In this case, for example, the surgical video quality control unit 105 reduces, as indicated in a balloon on the right side of FIG. 7, the bit rate of the surgical video P2 that is the secondary video to 25 Mbps, and the surgical video P1 and a surgical video P2-1 the bit rate of which has been reduced are transmitted to the reception device 3 via the information processing server 2.

This configuration enables real-time transmission of two surgical videos while maintaining the quality of the primary video.
Second specific example (specific example of tenth quality control described above)

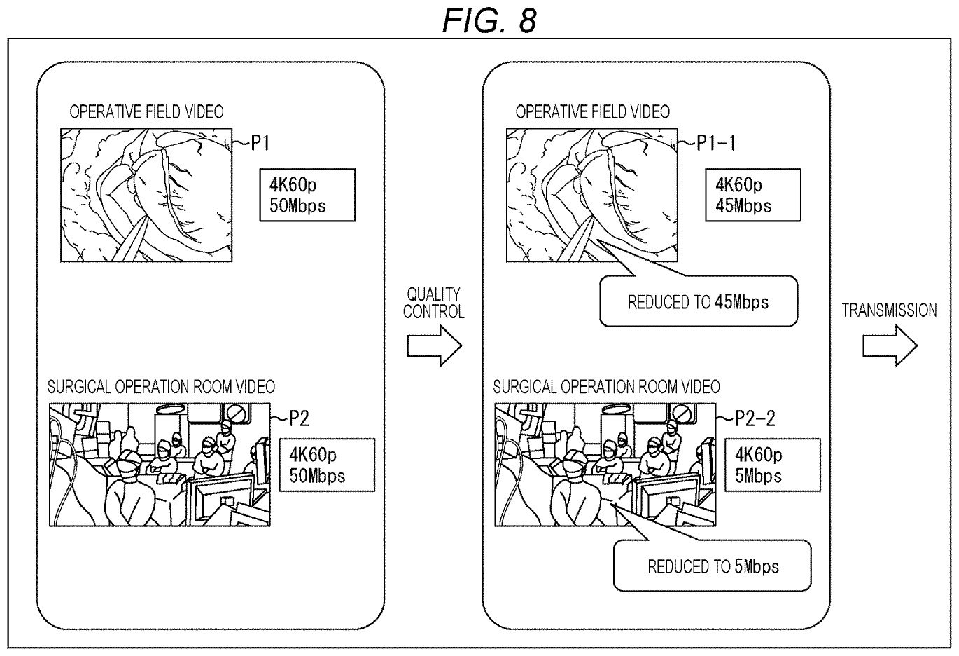

FIG. 8 is a diagram illustrating a second specific example of quality control of a surgical video.

In this example, it is assumed that the line status analysis unit 201 has determined each transmission rate to be 75 Mbps as the status of the communication line between the information processing server 2 and the surgical operation room system 1 and as the status of the communication line between the information processing server 2 and the reception device 3.

Furthermore, as illustrated on the left side of FIG. 8, it is assumed that the surgical video P1 and the surgical video P2 have been acquired by the surgical video acquisition unit 102. The surgical video P1 and the surgical video P2 each have the resolution of 4K, the frame rate of 60 p, and the bit rate of 50 Mbps.

Since the sum of the bit rate of the surgical video P1 and the bit rate of the surgical video P2 is larger than the transmission rate between the three places, it is impossible, in this situation, to transmit the surgical video P1 and the surgical video P2 while keeping the original quality.

In a case where the surgical video P2 is analyzed by the surgical video analysis unit 103 to be a surgical video having a lower importance than the surgical video P1, the surgical video quality control unit 105, for example, reduces the bit rate of the surgical video P1 to 45 Mbps as illustrated in the upper right balloon of FIG. 8 and reduces the bit rate of the surgical video P2 to 5 Mbps to control the quality of the two surgical videos. A surgical video P1-1 and a surgical video P2-2 the total bit rate of which is 50 Mbps that is lower than the transmission rate are transmitted to the reception device 3 via the information processing server 2.

This configuration enables real-time transmission of two surgical videos while maintaining the quality of the surgical video P1 showing an important scene.
Third specific example (specific example of the combination of the second, sixth, and tenth quality control described above)

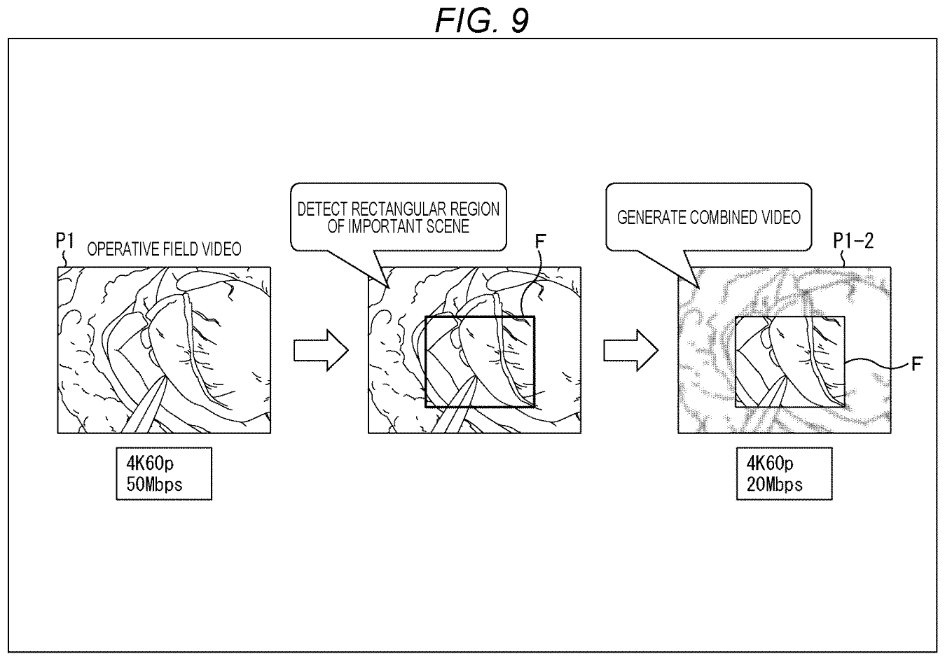

FIG. 9 is a diagram illustrating a third specific example of quality control of a surgical video.

In this example, it is assumed that the line status analysis unit 201 has analyzed each transmission rate to be 50 Mbps as the status of the communication line between the information processing server 2 and the surgical operation room system 1 and as the status of the communication line between the information processing server 2 and the reception device 3.

Furthermore, it is assumed that the surgical video P1 and the surgical video P2 described with reference to FIG. 8 have been acquired by the surgical video acquisition unit 102. The surgical video P1 and the surgical video P2 each have the resolution of 4K, the frame rate of 60 p, and the bit rate of 50 Mbps. FIG. 9 only illustrates the processing on the surgical video P1.

Since the sum of the bit rate of the surgical video P1 and the bit rate of the surgical video P2 is larger than the transmission rate between the three places, it is impossible, in this situation, to transmit the surgical video P1 and the surgical video P2 while keeping the original quality.

Since the surgical video P1 is showing the ligation of the important blood vessel, the surgical video P1 is analyzed to be a surgical video with higher importance, and in a case where the user selects a region including the important blood vessel as a region desired to be viewed in high quality, the surgical video analysis unit 103 detects a rectangular region (a region defined by a frame F) including the important blood vessel and the periphery thereof as indicated by a balloon at the center of FIG. 9.

The surgical video quality control unit 105 cuts out the rectangular region detected by the surgical video analysis unit 103 from the surgical video P1 by cropping. Furthermore, the surgical video quality control unit 105 combines the video of the rectangular region cut out by cropping with the video obtained by reducing the resolution of the entire surgical video P1 to have a reduced bit rate of 5 Mbps, and generates a surgical video P1-2 that is a single combined video as illustrated on the right side of FIG. 9. The bit rate of the surgical video P1-2 is, for example, 20 Mbps. On the right side of FIG. 9, blurring the region other than the rectangular region defined by the frame F indicates that the resolution of the region other than the rectangular region is low.

The important region may be selected on the surgical operation room server 11 side by analyzing the video instead of being selected by the user as described above.

The quality of the surgical video P2 is controlled by reducing the bit rate to 5 Mbps and the surgical video P2-2 is generated, similarly to the case described with reference to FIG. 8.

The surgical video P1-2 and the surgical video P2-2 the total bit rate of which is 25 Mbps that is lower than the transmission rate are transmitted to the reception device 3 via the information processing server 2.

This configuration enables real-time transmission of two surgical videos while maintaining the quality of the important region of the surgical video P1.

### Modifications

The surgical video quality control unit 105 may be implemented in the information processing server 2, and the quality of the surgical video may be controlled in the information processing server 2. In this case, the information output from each of the line status acquisition unit 101, the surgical video analysis unit 103, and the video selection status reception unit 104 is transmitted to the information processing server 2.

Although the quality is controlled by reducing at least one of the resolution and the frame rate, the quality of the surgical video may be controlled by another method. For example, the quality may be reduced by switching the scanning method of the surgical video from the progressive scanning to the interlace scanning.

Although the case where the video to be distributed is the surgical video has been described, the above-described technology is also applicable to a case of distributing various videos such as a video captured by a monitoring camera.
About program

The series of processing described above can be executed by hardware or software. In a case where the series of processing is executed by software, a program included in the software is installed from a program recording medium to a computer incorporated in dedicated hardware, a general-purpose personal computer, or the like.

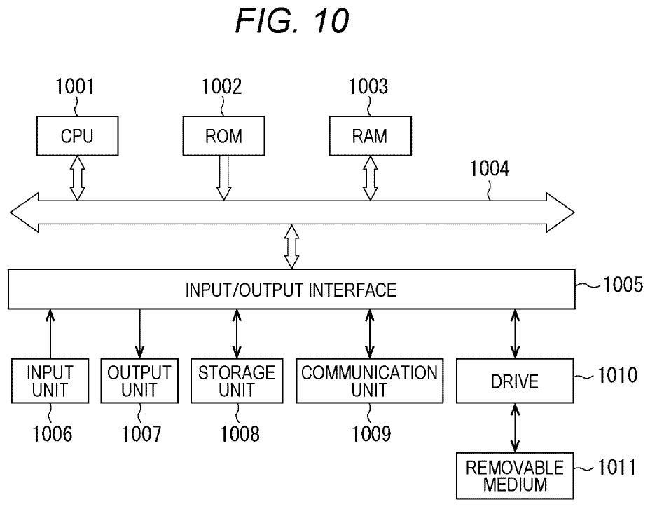

FIG. 10 is a block diagram illustrating a configuration example of hardware of a computer that executes the above-described series of processing by a program. Each of the information processing server 2, the reception device 3, and the surgical operation room server 11 has a configuration similar to the configuration illustrated in FIG. 10.

A central processing unit (CPU) 1001, a read only memory (ROM) 1002, and a random access memory (RAM) 1003 are interconnected via a bus 1004.

An input/output interface 1005 is further connected to the bus 1004. An input unit 1006, an output unit 1007, a storage unit 1008, a communication unit 1009, and a drive 1010 are connected to the input/output interface 1005. The drive 1010 drives a removable medium 1011 such as a magnetic disk, an optical disk, a magneto-optical disk, or a semiconductor memory.

In the computer configured as described above, for example, the CPU 1001 loads a program stored in the storage unit 1008 into the RAM 1003 via the input/output interface 1005 and the bus 1004 and executes the program to perform the above-described series of processing.

For example, the program to be executed by the CPU 1001 is recorded in the removable medium 1011 or provided via a wired or wireless transmission medium such as a local area network, the Internet, or a digital broadcast, and installed in the storage unit 1008.

Note that the program executed by the computer may be a program for performing processes in time series in the order described in the present specification, or may be a program for performing processes in parallel or at necessary timing such as when a call is made.

In the present specification, a system is intended to mean a set of a plurality of components (devices, modules (parts), or the like), and it does not matter whether or not all the components are disposed in the same housing. Therefore, a plurality of devices stored in different housings and connected via a network and one device in which a plurality of modules is stored in one housing are both systems.

The effects described in the specification are merely examples and are not limited, and other effects may be provided.

Embodiments of the present technology are not limited to the above-described embodiments, and various modifications may be made without departing from the gist of the present technology.

For example, the present technology may be embodied in cloud computing in which a function is shared and executed by a plurality of devices via a network.

Furthermore, each step described in the flowchart described above can be performed by one device or by a plurality of devices in a shared manner.

Furthermore, in a case where a plurality of processes is included in one step, the plurality of processes included in the one step may be performed by one device, or may be performed by a plurality of devices in a shared manner.
Examples of combinations of configurations The present technology can have the following configurations.
   (1)

An information processing method performed by an information processing system, the information processing method including:
controlling, on the basis of a selection status of a video by a user viewing a plurality of surgical videos using a reception device, the plurality of surgical videos including a video captured in a surgical operation room, and a status of a communication line, quality of each of a plurality of the surgical videos; and
transmitting the surgical videos the quality of which has been controlled to the reception device via an information processing server on a network.
   (2)

The information processing method according to (1) above, further including
reducing quality of the surgical video other than the surgical video selected by the user.
   (3) The information processing method according to (1) or (2) above, further including
reducing quality of the surgical video by cutting out a region selected by the user.
   (4)

The information processing method according to any one of (1) to (3) above, in which
quality of the surgical video is controlled such that, on the basis of priority set according to selection by the user, the surgical video the priority of which is high has a higher quality than the surgical video the priority of which is low.
   (5)

The information processing method according to any one of (1) to (4) above, in which
quality of the surgical video is controlled by changing at least one of resolution and a frame rate.
   (6)

The information processing method according to any one of (1) to (5) above, in which
quality of the surgical video is controlled by not transmitting the surgical video other than a certain number of the surgical videos having a high priority set according to selection by the user.
   (7) The information processing method according to any one of (1) to (6) above, in which
quality of a plurality of the surgical videos is controlled by combining a certain number of the surgical videos having a high priority set according to selection by the user into a single video.
   (8)

The information processing method according to any one of (1) to (7) above, further including
setting priority of each of a plurality of the surgical videos on the basis of medical device information included in attribute information of each of the surgical videos, in which
quality of the surgical video is controlled on the basis of the priority.
   (9)

The information processing method according to any one of (1) to (8) above, further including
analyzing content of the surgical video, in which
quality of the surgical video is controlled according to an analysis result of the content of the surgical video.
   (10)

The information processing method according to (9) above, in which
quality of the surgical video is controlled on the basis of the analysis result of the content of the surgical video and the status of the communication line.
   (11)

The information processing method according to any one of (1) to (10) above, further including acquiring information indicating a selection status of the surgical video by the user, the information being transmitted from the reception device.

(12)

The information processing method according to any one of (1) to (11) above, further including
acquiring a status of a communication line between a medical facility in which the surgical operation room is located and the information processing server that distributes the surgical video uploaded from the medical facility, and a status of a communication line between the information processing server and the reception device.
   (13)

An information processing system including
a quality control unit that controls, on the basis of information indicating a selection status of a video by a user viewing a plurality of surgical videos using a reception device, the plurality of surgical videos including a video captured in a surgical operation room, and a status of a communication line, quality of each of a plurality of the surgical videos, and
a transmission control unit that transmits the surgical videos the quality of which has been controlled to the reception device via an information processing server on a network.
   (14)

A program that causes a computer to execute processing of
controlling, on the basis of information indicating a selection status of a video by a user viewing a plurality of surgical videos using a reception device, the plurality of surgical videos including a video captured in a surgical operation room, and a status of a communication line, quality of each of a plurality of the surgical videos, and
transmitting the surgical videos the quality of which has been controlled to the reception device via an information processing server on a network.

### REFERENCE SIGNS LIST

1 Surgical system
2 Information processing server
3 Reception device
11 Surgical operation room server
101 Line status acquisition unit
102 Surgical video acquisition unit
103 Surgical video analysis unit
104 Video selection status reception unit
105 Surgical video quality control unit
106 Surgical video transmission unit
201 Line status analysis unit
202 Surgical video reception unit
203 Surgical video transmission unit
301 Surgical video reception unit
302 Surgical video display unit
303 Video selection status transmission unit

1. An information processing method performed by an information processing system, the information processing method comprising:
controlling, on a basis of a selection status of a video by a user viewing a plurality of surgical videos using a reception device, the plurality of surgical videos including a video captured in a surgical operation room, and a status of a communication line, quality of each of a plurality of the surgical videos; and
transmitting the surgical videos the quality of which has been controlled to the reception device via an information processing server on a network.

2. The information processing method according to claim 1, further comprising
reducing quality of the surgical video other than the surgical video selected by the user.

3. The information processing method according to claim 1, further comprising
reducing quality of the surgical video by cutting out a region selected by the user.

4. The information processing method according to claim 1, wherein
quality of the surgical video is controlled such that, on a basis of priority set according to selection by the user, the surgical video the priority of which is high has a higher quality than the surgical video the priority of which is low.

5. The information processing method according to claim 1, wherein
quality of the surgical video is controlled by changing at least one of resolution and a frame rate.

6. The information processing method according to claim 1, wherein
quality of the surgical video is controlled by not transmitting the surgical video other than a certain number of the surgical videos having a high priority set according to selection by the user.

7. The information processing method according to claim 1, wherein
quality of a plurality of the surgical videos is controlled by combining a certain number of the surgical videos having a high priority set according to selection by the user into a single video.

8. The information processing method according to claim 1, further comprising
setting priority of each of a plurality of the surgical videos on a basis of medical device information included in attribute information of each of the surgical videos, wherein
quality of the surgical video is controlled on a basis of the priority.

9. The information processing method according to claim 1, further comprising
analyzing content of the surgical video, wherein
quality of the surgical video is controlled according to an analysis result of the content of the surgical video.

10. The information processing method according to claim 9, wherein
quality of the surgical video is controlled on a basis of the analysis result of the content of the surgical video and the status of the communication line.

11. The information processing method according to claim 1, further comprising
acquiring information indicating a selection status of the surgical video by the user, the information being transmitted from the reception device.

12. The information processing method according to claim 1, further comprising
acquiring a status of a communication line between a medical facility in which the surgical operation room is located and the information processing server that distributes the surgical video uploaded from the medical facility, and a status of a communication line between the information processing server and the reception device.

13. An information processing system comprising:
a quality control unit that controls, on a basis of information indicating a selection status of a video by a user viewing a plurality of surgical videos using a reception device, the plurality of surgical videos including a video captured in a surgical operation room, and a status of a communication line, quality of each of a plurality of the surgical videos; and
a transmission control unit that transmits the surgical videos the quality of which has been controlled to the reception device via an information processing server on a network.

14. A program that causes a computer to execute processing of:
controlling, on a basis of information indicating a selection status of a video by a user viewing a plurality of surgical videos using a reception device, the plurality of surgical videos including a video captured in a surgical operation room, and a status of a communication line, quality of each of a plurality of the surgical videos; and
transmitting the surgical videos the quality of which has been controlled to the reception device via an information processing server on a network.
